(19) United States
(12) Patent Application Publication
(10) Pub. No.: US 2009/0242075 A1
(43) Pub. Date: Oct. 1, 2009

(54) BOTTLE FILLER

(75) Inventors: Louis M. Busick, Westerville, OH (US); Kevin R. Seeley, Delaware, OH (US); Galen L. Gerig, Columbus, OH (US)

Correspondence Address: STANDLEY LAW GROUP LLP, 6300 Riverside Drive, Dublin, OH, 43017, US

(73) Assignee: Zohar Waterworks, LLC, Charlotte, NC (US)

(21) Appl. No.: 12/413,578

(22) Filed: Mar. 29, 2009

Related U.S. Application Data
(60) Provisional application No. 61/041,096, filed on Mar. 31, 2008.

(51) Int. Cl. B67D 5/37; B67D 5/58; B67D 5/64; B67D 5/66

(52) U.S. Cl. 141/85; 141/372; 141/373; 141/379

(57) ABSTRACT

A self sanitizing bottle filler supplying filtered water. The bottle filler utilizes UV light to create ozone in a sealed dispensing chamber after each use. The UV light and ozone sanitize the interior surfaces of the dispensing mechanism and water delivery components. The bottle filler encloses the outlet spout after each use to prevent airborne contamination. The water stream may be illuminated helping users to position a container, preventing spills and splash back.

### TECHNICAL FIELD

This application claims the benefit of U.S. Provisional Application No. 61/041,096, filed Mar. 31, 2008, which is hereby incorporated by reference in its entirety.

### TECHNICAL FIELD

The present invention relates to water bottle filler. More particularly, the present invention relates a water bottle filler providing water dispensing nozzle sanitization.

### BACKGROUND AND SUMMARY OF THE INVENTION

In recent years, the popularity of bottled drinking water has increased. This increase may be attributed to several factors, the portability of the bottled water, the perceived taste of bottled water, and concerns over germs and bacteria associated with public water fountains.

The bottle filler described herein provides an environmentally friendly alternative to bottle water. By providing users with the ability to reuse containers the bottle filler reduces non-biodegradable plastic waste and decreases dependency on natural resources to produce containers. The large bottle alcove allows a large variety of containers to be used. The bottle filler may be adapted to fit almost any size of container a user desires to fill with water. Most embodiments of the bottle filler may accommodate 1 liter containers. In addition to providing users the ability to refill water containers, the bottle filler provides great tasting water through the use of filtration systems.

The bottle filler may employ a full line of filters and UV to help disinfect and remove impurities from the water. These filtration steps help improve the quality and taste of the water, as well as, providing a healthier alternative to most bottled water.

Another problem associated with public water sources is the concern over germs and bacteria. The dispensing nozzle of most water fountains is in constant contact with the air, and as such, in contact with any airborne germs or bacteria. The water fountain industry has paid little attention to this type of contamination. The bottle filler described herein is specifically designed to combat this type of contamination, in a variety of ways.

The design of the bottle filler helps to prevent any splash back that may occur during the filling process. By placing the outlet spout above the bottle alcove, only minimal splash back occurs. In addition, the bottle filler's outlet spout is not exposed to the outside air between uses. After an individual finishes filling a container. An electronic controller instructs a solenoid to close a dispenser door. This door creates a seal with the dispenser chamber, enclosing the dispenser nozzle. To further prevent contact contamination the bottle filler is designed so that the dispenser nozzle and the container to be filled may never come into contact. This ensures that contaminants on the container are not transferred to the nozzle.

Lastly, the bottle filler sterilizes the nozzle between uses. This sterilization ensures that any splash back that reached the nozzle will not contaminate the water dispensed during the next use. The bottle filler uses ultraviolet (“UV”) light and ozone to sterilize the dispensing nozzle between each use. A UV bulb is placed in the dispensing chamber. After a user releases the activation button, the dispenser door closes sealing the dispensing chamber; trapping air and any remaining water in the chamber. The UV light bathes the entire chamber and associated components. The UV light sanitizes the surface of the chamber and components, including but not limited to, the outlet spout and the outlet spout cover.

The UV light also converts the oxygen in the trapped air inside the dispensing chamber into ozone. Ozone is an excellent sanitizer and further sanitizes the water dispensing surfaces. After the ozone is created it may settle toward the lower portion of the dispensing chamber surrounding the outlet spout and outlet spout cover. The design of the outlet spout cover allows this settling ozone to encompass the entire outlet spout sanitizing its entire surface. This ozone sanitization ensures no bacteria or organisms from air exposure during use and from any potential splash back remain, and in turn that no contamination is passed to the next user of the bottle filler.

### BRIEF DESCRIPTION OF THE DRAWINGS

In addition to the features mentioned above, other aspects of the exemplary embodiments will be readily apparent from the following descriptions of the drawings and exemplary embodiments, wherein like references numerals across the several views refer to identical or equivalent features, and wherein:

FIG. 1 is a front view of an exemplary embodiment of a bottle filler.

FIG. 2 is a perspective view of an exemplary embodiment of a bottle filler dispensing liquid.

FIG. 3 is a top perspective view of an exemplary embodiment of a bottle filler.

FIG. 4 is a perspective view of an exemplary embodiment of a bottle filler in conjunction with a water fountain.

FIG. 5 is a perspective view of an exemplary embodiment of a wall mounted bottle filler.

FIG. 6 is a perspective view of an exemplary embodiment of the interior of a bottle filler.

FIG. 7 is a perspective view of an exemplary embodiment of the interior of a bottle filler having a closed dispensing door.

FIG. 8 is a perspective view of an exemplary embodiment of the interior of a bottle filler having an open dispensing door.

FIG. 9 is a front view of an exemplary embodiment of a bottle filler having a payment system.

FIG. 10 is a cross section view of an exemplary embodiment of a bottle filler having an open dispensing door.

FIG. 11 is a cross section view of an exemplary embodiment of a bottle having a closed dispending door.

### DETAILED DESCRIPTION OF THE EXEMPLARY EMBODIMENT(S)

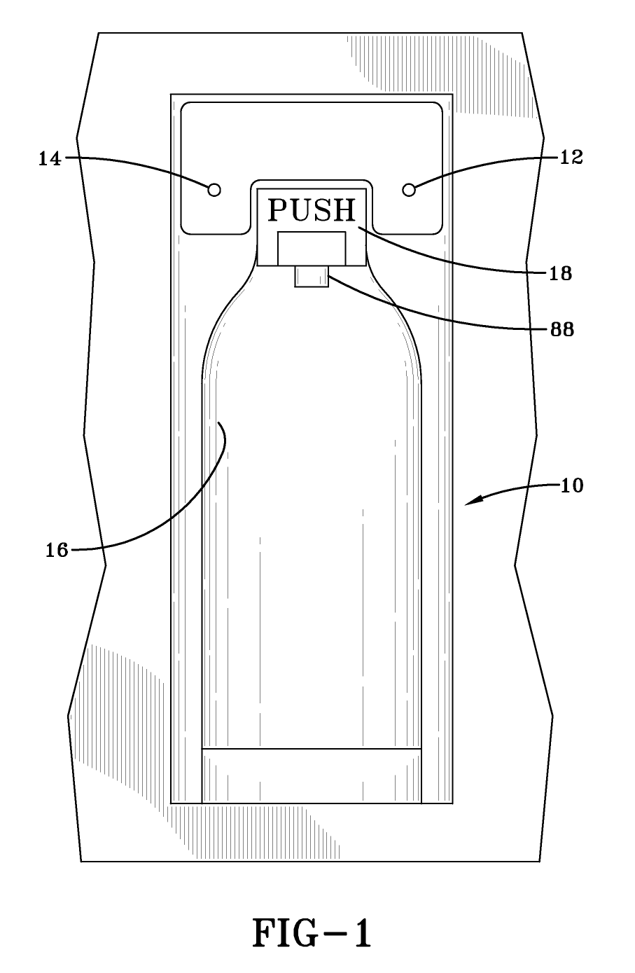

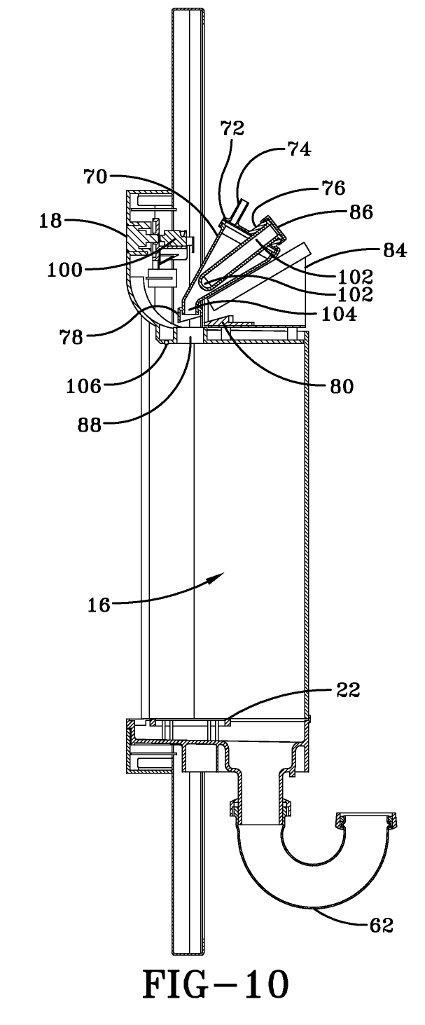

FIG. 1 is a front view of an exemplary embodiment of a bottle filler 10. A filter indicator 12 may be located at any position on the bottle filler wherein it may be visible to a user or technician. The filter indicator 12 provides a visual indication regarding the functional status of any filtration system associated with the bottle filler 10. Likewise, an ultraviolet (“UV”) indicator 14 may also be located at any position on the bottle filler 10 wherein it may be visible to a user or technician. The UV indicator 14 provides a visual indication regarding the functional status of the UV bulb 102 (shown in FIG. 10) within the bottle filler 10. The filter indicator 12 and UV indicator 14 may be a light or other device capable of giving a visual queue, such as an LED.

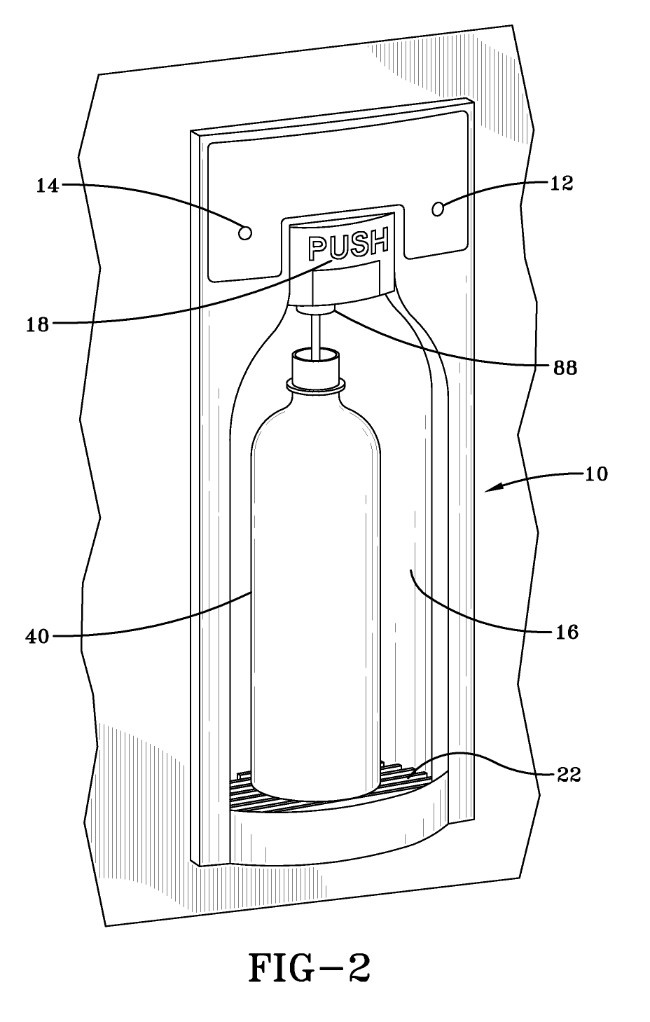

The front of the bottle filler 10 may define a recessed bottle alcove 16. The bottle alcove 16 may be adapted to receive a container 40 (shown in FIG. 2), examples of containers 40 that may be placed in the bottle alcove 16 include, but are not limited to, bottles, cups, carafes, pitchers, or other containers 40 suitable for containing a liquid. More specifically, the bottle alcove may be adapted to receive containers 40 that hold at least one liter of liquid. In still other embodiments, the bottle alcove 16 may be configured to receive containers 40 that hold over one liter of liquid. An aperture is provided in the top portion of the bottle alcove forming a fluid guide 88 to deliver water to a liquid container 40. The fluid guide 88 may be positioned to allow easy alignment with a liquid container 40 to be filled.

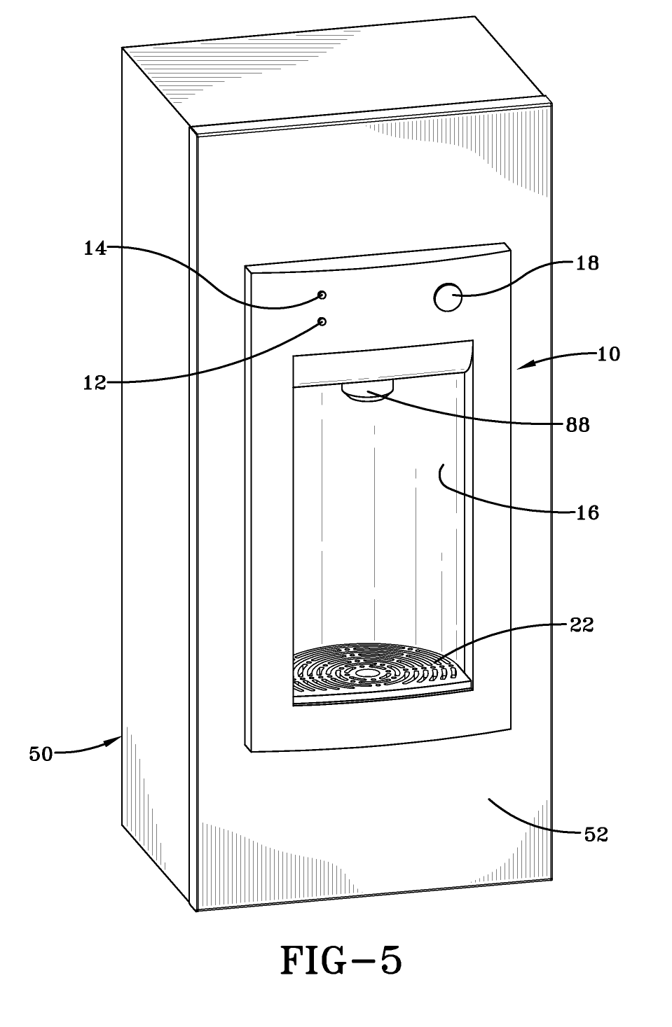

The bottle filler 10 may also include an activation button 18 on a front surface thereof. The activation button 18 is positioned to be easily accessible to a user. When a user engages the activation button 18, liquid is dispensed from the bottle filler 10. Upon release of the activation button 18, the flow of liquid from the bottle filler 10 is stopped and the sanitation process begins. In other exemplary embodiments, the filter indicator 12, UV indicator 14, and activation button 18 may be located on a front plate 52 (shown in FIG. 5).

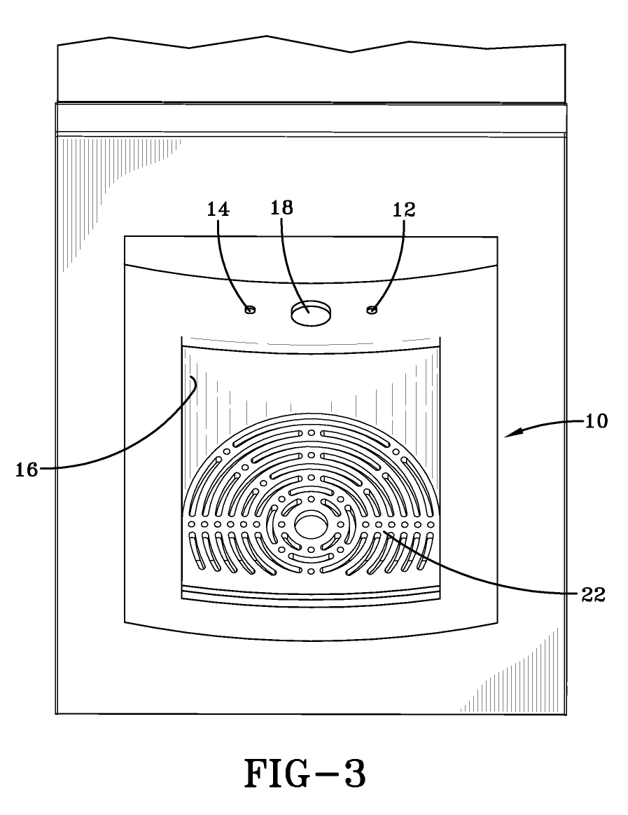

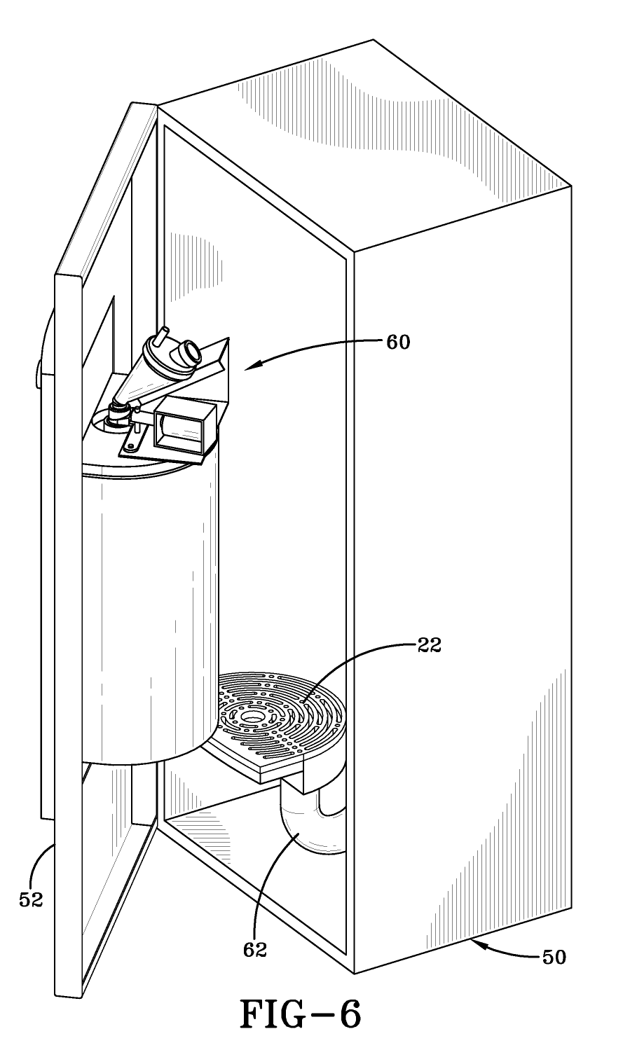

FIG. 2 is a perspective view of an exemplary embodiment of a bottle filler 10 having a liquid container 40 in the bottle alcove 16. A grille 22, disposed within the bottle alcove 16, provides a platform on which the container 40 to be filled may be placed. As shown FIG. 3, the grille 22 may be slotted allowing liquid to pass through the grille and into an attached drainage pipe 62 (shown in FIG. 6). The slotted grille 22 prevents spilled liquid from pooling in the bottle alcove 16 where bacteria and other contaminants may collect. The grille 22 may also be illuminated from a light source located above or below the grille. The illumination may provide a target area on which to place a liquid container 40 to assist in alignment of the liquid container 40 with the liquid stream from the fluid guide 88 during operation. The grille 22 may be permanently fixed inside the bottle alcove 16 or detachably mounted to provide easy cleaning and access to the drain pipe 62.

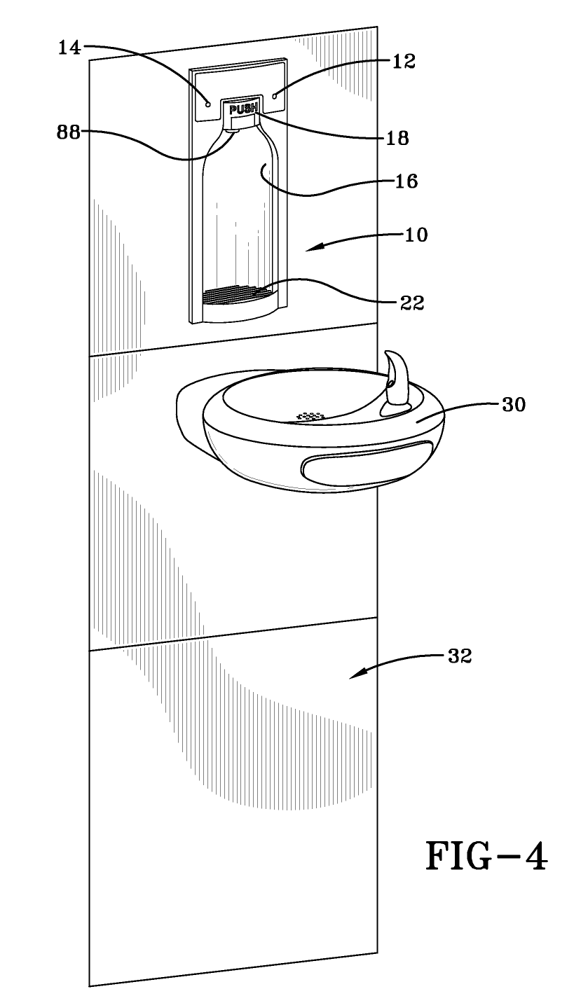

FIG. 4 is a perspective view of an exemplary embodiment of a bottle filler 10. As shown FIG. 4, the bottle filler 10 may be built into a wall 32 adjacent to a standard water fountain 30. The in-wall embodiment decreases the amount of space needed for the bottle filler 10. In other exemplary embodiments, the bottle filler may be mounted to a wall. The wall mounted bottle filler 10 allows retrofit installation of the bottle filler 10. Both the in-wall and wall mounted bottle filler 10 embodiments may be adapted for use with wall studs having 16 inch centers, 20 inch centers, or any other stud configurations.

A Bottle filler 10 placed adjacent to a water fountain 30 may share the same supply line filtration system or maintain independent supply line filters (not shown in the Figures). The supply line filtration system may be located behind the wall 32 accessible by a locking panel to prevent unauthorized access. In other exemplary embodiments, the supply line filtration system may be exposed and wall mounted. In still other exemplary embodiments, the supply line filtration system may be contained with the casing 50 (shown in FIG. 5).

FIG. 5 illustrates an exemplary embodiment of a bottle filler 10 housed in a casing 50. In some embodiments, the casing 50 may be adapted to mount to a wall for easy retrofit installation. In a wall mounted embodiment, the casing 50 may be fixed to a wall 32 by mechanical fasteners, adhesive, or other suitable means. In other embodiments, the casing 50 may be adapted for insertion into a wall for in-wall installation. The casing 50 includes a front plate 52 having hinges on one side and a latching mechanism on an opposing side thereof. The front plate 52 opens allowing access to the dispensing mechanism 60, as illustrated in FIG. 6.

In the embodiment shown in FIG. 6, the grille 22 is mounted to the drain pipe 62 and remains stationary as the casing 50 is opened. The drain pipe 62 may be a one and a half inch stub connection or any other suitable size to accommodate waste fluid from the bottle filler 10. As mentioned above, the supply line filtration system may also be enclosed in the casing 50 and accessible by the front plate 52.

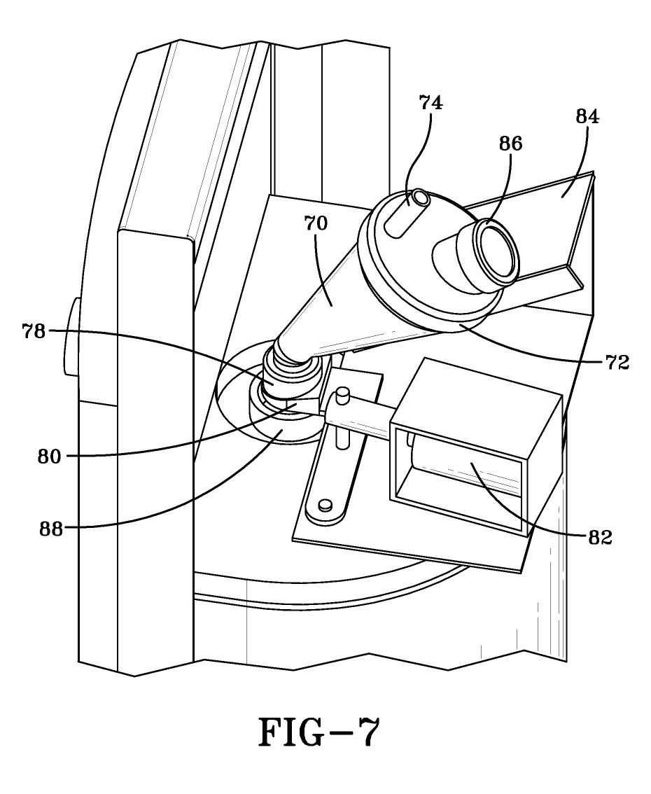

The dispensing mechanism 60 of the exemplary bottle filler 10 is depicted in FIG. 7. The dispensing mechanism 60 includes a liquid retention vessel 70 having a liquid retention cap 72 attached thereto, a liquid inlet 74 extending from the liquid retention cap, a UV bulb chamber 76 (shown in FIG. 10) extending into the liquid retention vessel through the liquid retention cap, an outlet spout 104 (shown in detail in FIG. 10), a outlet spout cover 78 affixed to the outlet spout, a dispenser door 80 in complementary engagement with the outlet spout cover, a dispenser door solenoid 82 in communication with the dispenser door, and a liquid solenoid valve (not shown in the Figures). The dispensing mechanism 60 may be constructed with UV resistant plastics or other suitable materials including, but not limited to, stainless steel. A vessel support 84 may be affixed atop the bottle alcove 16 and is adapted to support the retention vessel 70.

The liquid retention cap 72 is affixed to a top portion of the retention vessel 70 forming an air tight seal between the liquid retention cap 72 and the liquid retention vessel 70. In other exemplary embodiments, the liquid retention cap 72 and the liquid retention vessel 72 may have a unitary design. The liquid inlet 74 extends from the liquid retention cap 72 and is adapted to connect to the fluid supply line (not shown in the Figures). The UV chamber 76 is removably inserted into the liquid retention vessel 70 through an aperture in the liquid retention cap 72. The UV chamber 76 has a hollow body adapted to retain at least one UV bulb 102 (shown in FIG. 10). The UV chamber 76 allows a substantial portion of the UV bulb 102 to be located within the liquid retention vessel 70. In this manner, UV light from the at least one UV bulb 102 substantially fills the liquid retention vessel 70 interior. The UV chamber 76 may be constructed of optical quartz allowing the UV radiation produced by the at least one UV bulb 102 to escape the UV chamber and fill the liquid retention vessel 70. In other embodiments, the liquid retention cap 72 may be adapted to receive a UV bulb 102. This embodiment eliminates the need for a separate UV chamber 76.

The exemplary embodiment of the liquid retention vessel 70, illustrated in FIG. 7 has a substantially conical shape with a top portion having a larger diameter then a lower portion. The lower portion of the liquid retention vessel 70 may be turned downward forming an outlet spout 104. An outlet spout cover 78 extends from the lower portion of the liquid retention vessel 70 downward past the outlet spout 104. The end of the outlet spout cover 78 extending downward past the outlet spout 104 has an angled contact surface for engagement with a dispenser door 80. In some exemplary embodiments, the fluid retention vessel 70, the outlet spout 104, and the outlet spout cover 78 are a unitary design. In other embodiments, the fluid retention vessel 70, the outlet spout 104, and the outlet spout cover 78 may be independent components forming air tight seals therebetween.

The dispenser door 80 is slidably attached to the vessel support 84. In embodiments having no vessel support 84, the dispenser door 80 is affixed to the top portion of the bottle alcove 16. The dispenser door 80 includes an angled portion adapted to engage the angled portion of the outlet spout cover 78 forming an air tight seal. When the dispenser door 80 is engaged with the outlet spout cover 78 (closed position), fluid is prevented from passing through the liquid guide 88 in the top portion of the bottle alcove 16. The extension of the outlet spout cover 78 downward past the outlet spout 104 prevents the contaminating contact between the outlet spout 78 and the dispenser door 80.

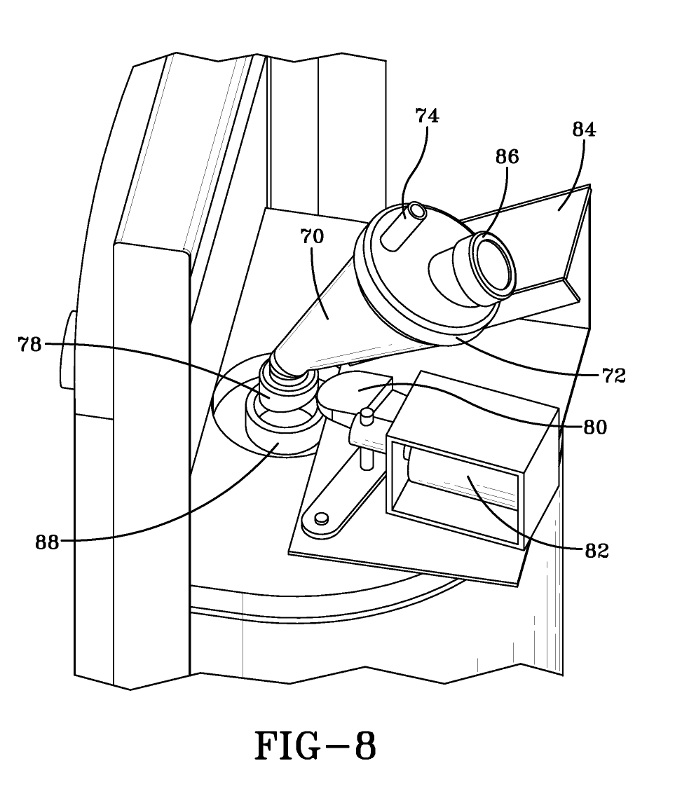

When the dispenser door 80 is disengaged from the outlet spout 78 liquid (open position) liquid is allowed to flow from the liquid retention vessel 70 through the outlet spout 104 and into the liquid guide 88, as illustrated in FIG. 8. The dispenser door 80 is moved from the open position to the closed position and vice versa by the dispenser door solenoid 82 in mechanical communication with the dispenser door. The dispenser door solenoid 82 is supported by the vessel support 84. In embodiments wherein a vessel support 84 is not present, the dispenser door solenoid 82 may be adapted to attach to the top portion of the bottle alcove 16.

The liquid retention cap 72 may be adapted to receive a bulb chamber cap 86 locking the UV bulb chamber 76 into place and forming an air tight seal around the UV bulb chamber 76. In other embodiments, the liquid retention cap 72 may be adapted to firmly hold the UV bulb chamber 76 creating an air tight seal and eliminating the need for a bulb chamber cap 86. In still other exemplary embodiments, the bulb chamber cap 86 is adapted to fit over the UV bulb 102 creating an air tight seal.

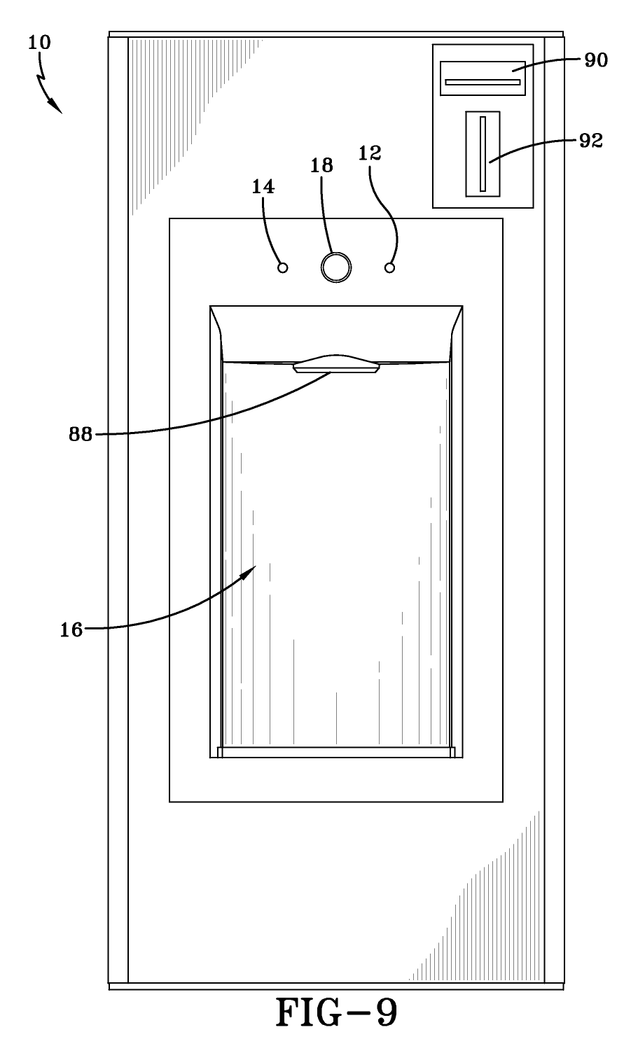

FIG. 9 illustrates an exemplary embodiment of a bottle filler 10 having a payment system in the form of a currency accepter 90 and a card reader 92, on a front surface thereof. Embodiments including a payment system require a user to pay a predetermined amount of money or credits before a metered amount of fluid will be dispensed. The currency accepter may accept currency in either paper or coin form. The card reader may accept debit cards, credit cards, or any other suitable similar cards.

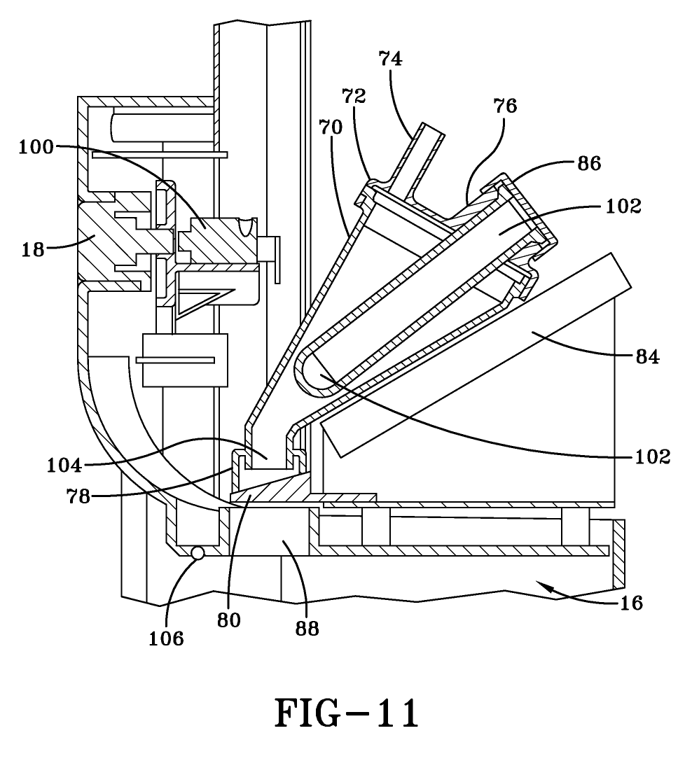

The bottle filler 10 according to the present invention is basically constructed as described above. Operation and additional embodiments of the bottle filler 10 will be described below with reference to FIGS. 10 and 11, wherein FIG. 10 illustrates the dispenser door 80 in an open position and FIG. 11 illustrates the dispenser door in a closed position.

To begin dispensing fluid, a user presses the activation button 18 in communication with a controller 100. The controller 100 may be battery powered or wired directly to a power source. In battery powered embodiments, the battery (not shown in the Figures) may be mounted inside the casing 50. The controller 100 is in communication with the dispenser door solenoid 82, UV Bulb 102, and the liquid solenoid valve. After the activation button 18 has been selected, the control 100 directs the dispenser door solenoid 82 to activate. The dispenser door solenoid 82 then disengages the dispenser door 80 from the outlet spout cover 78, moving the dispenser door into an open position, as illustrated in FIG. 10.

The controller 100 may then provides power to the UV bulb 102 illuminating the interior of the liquid retention vessel 70. In other embodiments, a separate UV light drive may be included. The controller 100 then directs the liquid solenoid valve to open allowing fluid from the supply line to pass through the fluid inlet 74 into the liquid retention vessel 70. Once inside the liquid retention vessel 70, the liquid may pass over and around the UV bulb chamber 76 or UV bulb 102. Exposing the liquid to UV light within the liquid retention vessel 70 removes contaminates from the liquid missed by the fluid supply filtration system. Gravity and the pressure from the supply line force the liquid downward through the outlet spout 104. The outlet spout 104 directs the liquid through the fluid guide 88 into an awaiting container 40 positioned in the bottle alcove 16.

To aid in the alignment of the container 40 with the liquid stream, the controller 100 may illuminate a light 106 located adjacent the fluid guide 88 in the bottle alcove 16. The light 106 may be an LED or other suitable lighting device. The positioning of the light 106 may be such that the emitted light illuminates the liquid stream exiting the fluid guide 88. In other embodiments, the light 106 may be directed at the grille 22 providing a target area for container 40 placement. In still other embodiments, multiple lights 106 may be arranged in the bottle alcove 16 and directed toward the fluid stream and the grille 22. A light may also be mounted inside the liquid retention vessel 70 directly above the outlet spout 104 directing the light downward so as to illuminate the light stream from within. The light 106 may be powered continuously or activated after the activation button 18 has been depressed.

After the container 40 has been filled to a desired level, the user releases the activation button 18. The release of the activation button 18, triggers the controller 100 to direct the liquid solenoid valve to cease the flow of liquid to the fluid inlet 74. The remaining liquid in the liquid retention vessel 70 empties through the outlet spout 104, leaving only air and possibly a small amount of liquid in the liquid retention vessel 70.

At a predetermined time after the liquid solenoid valve has been shut off, the controller 100 directs the dispenser door solenoid to seal the liquid retention vessel 70 by mating the dispenser door 80 to the outlet spout cover 78 (closed position). Air remaining in the retention vessel 70 becomes trapped therein. The time between the shut off the liquid supply line and the closing of the dispenser door 80 may be determined by the amount of time necessary for the majority of liquid to drain from the liquid retention vessel 70. This time may be dependent upon the size of the liquid retention vessel 70 and the distance of the supply line between the liquid inlet 74 and the liquid solenoid valve.

The controller 100 directs the UV bulb 102 to remain illuminated for a predetermined amount of time after the dispenser door 80 moves into the closed position. This allows the UV radiation from the UV bulb 102 to convert a portion of the oxygen in the trapped air within the retention vessel 70 into ozone. Because the liquid retention vessel 70 is sealed the ozone cannot escape and remains in the liquid retention vessel 70 and sanitizes the interior of the liquid retention vessel 70, the outlet spout 104, and the interior of the outlet spout cover 78.

The controller 100 may direct the UV bulb 102 to remain illuminated for a time sufficient to allow substantially all of the bacteria and contaminating organisms to be killed or sterilized inside the liquid retention vessel 70, by way of UV radiation or ozone production. This illumination time may be dependent on the size of the liquid retention vessel 70 and the filtration of the liquid before entering the liquid retention vessel 70. In other embodiments, the UV bulb 102 may remain on for approximately 20 to 45 seconds after the dispenser door 80 has mated with the outlet spout cover 78.

In other exemplary embodiments, the liquid retention vessel 70 may be in a vertical configuration. A vertical configuration may allow for increased room in a casing 50. This configuration may also allow the light from the UV bulb 102 to illuminate the liquid stream. This illumination may aid in the alignment of a container 40 with the liquid stream.

In other exemplary embodiments, an LCD display (not shown in the Figures) may be located on the interior of the front plate 52. The LCD display may be used to program the bottle filler 10. The LCD display may also be used to provide a visual signal indicating any malfunction with the bottle filler 10. In other exemplary embodiments, the LCD display may be mounted inside the casing 50 (shown in FIG. 5). The LCD display may be used to set the time intervals involved in the dispensing process.

In other exemplary embodiment, the dispensing mechanism 60 may be replaced the supply line and the supply line solenoid. In this configuration, once the activation button 18 is engaged, the control 100 directs the supply line solenoid to allow water to flow through the fluid guide 88. After dispensing a desired amount of water the activation button 18 may be released and the controller 100 directs the supply line solenoid to stop the water flow through the bottle filler 10.

While certain exemplary embodiments are described in detail above, the scope of the invention is not to be considered limited by such disclosure, and modifications are possible without departing from the spirit of the invention.

What is claimed is:

1. An apparatus for providing potable water comprising:
a liquid retention vessel, said liquid retention vessel adapted to form an outlet spout;
a liquid retention cap affixed to said liquid retention vessel, said liquid retention cap adapted to form an air tight seal with said liquid retention vessel;
a liquid inlet extending upwardly from said liquid retention cap, said liquid inlet adapted to receive a liquid from a liquid supply line;
a UV bulb chamber passing through said liquid retention cap, said UV bulb chamber adapted to receive at least one UV bulb;
an outlet spout cover extending from said liquid retention vessel, said outlet spout cover extending downward beyond said outlet spout; and
a door having a closed position and an open position; wherein when said door is in said closed position, said door is in complementary engagement with said outlet spout cover, when said door is in said open position, said door is disengaged from said outlet spout cover.

2. The apparatus of claim 1, further comprising a solenoid in communication with said door, said solenoid adapted to move said door between said closed position and said open position.

3. The apparatus of claim 1, further comprising a vessel support adapted to support said liquid retention vessel.

4. The apparatus of claim 1, wherein said liquid retention vessel has a conical shape.

5. An apparatus for providing potable fluid comprising:
a bottle alcove for receiving a container to be filled, said bottle alcove including a bottom portion having a grille mounted thereto and an upper portion having a fluid guide disposed therein;
a vessel support mounted to said bottle alcove, said vessel support adapted to support a liquid retention vessel, said liquid retention vessel including a lower portion adapted to form an outlet spout aligned with said fluid guide;
a liquid retention cap affixed to said liquid retention vessel, said liquid retention cap adapted to receive a UV chamber, said UV chamber having a UV bulb disposed therein;
a liquid inlet extending upward from said liquid retention cap; said fluid inlet adapted to allow fluid to flow into said liquid retention vessel; and
an outlet spout cover extending downward from said liquid retention vessel past said outlet spout, said outlet spout cover adapted to complementary engage a dispenser door slidably attached to said vessel support, said dispenser door having a closed position and an open position, wherein when said dispenser door is in said closed position, said dispenser door is in complementary engagement with said outlet spout cover, when said door is in said open position, said door is disengaged from said outlet spout cover.

6. The apparatus of claim 5, further comprising a solenoid in communication with said dispenser door, said solenoid adapted to move said dispenser door between said closed position and said open position.

7. The apparatus of claim 5, wherein said liquid retention vessel has a conical shape.

8. The apparatus of claim 5, further comprising a light disposed within said bottle alcove, said light adapted to illuminate said bottle alcove.

9. The apparatus of claim 5, further comprising a drain pipe affixed to said grille.

10. The apparatus of claim 5, further comprising a UV chamber cap adapted to retain said UV chamber in said liquid retention cap.

11. An apparatus for providing potable water fluid comprising:
a casing, said casing including a hinged front plate;
a bottle alcove disposed within said front plate, said bottle alcove adapted to receive a container to be filled and having a fluid guide disposed therein;
a vessel support affixed to said bottle alcove, said vessel support adapted to receive and support a dispensing mechanism;
a UV light disposed within said dispensing mechanism, said UV light positioned so as to illuminate an interior of the dispensing mechanism;
a door adapted to seal said dispensing mechanism when in an extended position and unseal a said dispensing mechanism when in a retracted position, said door interposed between said dispensing chamber and said fluid guide when in a retracted position;
a door solenoid affixed to said vessel support adapted to extend and retract said door; and
a controller in communication with said door solenoid and said UV light, said controller adapted to control fluid supply to said dispensing mechanism;
whereby, when fluid is supplied to said dispensing chamber said controller directs said UV light to illuminate and directs said door solenoid to retract said door, allowing fluid to flow from said dispensing chamber through said fluid guide into said bottle alcove to fill said container;
wherein, when fluid supplied to said dispensing chamber is stopped, said controller directs said door solenoid to extend said door sealing said dispensing chamber said controller directs said UV light to remain illuminated for time sufficient to create ozone and to sterilize said interior of said dispensing chamber.

12. The apparatus of claim 11, wherein said dispensing chamber further includes:
a liquid retention vessel affixed to said vessel support; said liquid retention having a lower portion adapted to form an outlet spout;
a liquid retention cap affixed to said liquid retention vessel, said liquid retention cap adapted to form an air tight seal between said liquid retention cap and said liquid retention vessel;
a liquid inlet extending upward from said liquid retention cap; said fluid inlet adapted to provide fluid to said liquid retention vessel; and
an outlet spout cover extending downward past said outlet spout from said liquid retention vessel; said outlet spout cover adapted to seal said liquid retention vessel when engaged with said door.

13. The apparatus of claim 11, wherein said door is slidably mounted to said vessel support.

14. The apparatus of claim 11, further comprising a light mounted in said bottle alcove.

15. The apparatus of claim 11, further comprising a payment system, wherein said payment system is adapted to dispense a metered amount of fluid from said apparatus upon payment.

16. The apparatus of claim 11, further comprising an activation button disposed onto said front plate, said activation button in communication with said controller and adapted to activate said controller.

17. The apparatus of claim 11, wherein said bottle alcove is adapted to receive at least a 1 liter container.

18. The apparatus of claim 11, further comprising a grille located in a bottom portion of said bottle alcove, said grille affixed to a drain pipe

19. The apparatus of claim 11, further comprising a filter indicator and a UV indicator disposed on said front plate.

20. The apparatus of claim 11, further comprising a light disposed with said dispensing mechanism, said light adapted and positioned to illuminate a fluid as it exits said dispensing mechanism.
